(19) United States
(12) Patent Application Publication
(10) Pub. No.: US 2020/0022666 A1
(43) Pub. Date: Jan. 23, 2020

(54) SYSTEM AND METHOD FOR PROCESSING DATA FROM COMPUTED TOMOGRAPHY SCANS

(71) Applicant: R.A.W. S.R.L., Busto Arsizio (Varese) (IT)

(72) Inventors: Marco SOLBIATI, BUSTO ARSIZIO (IT); Katia PASSERA, BUSTO ARSIZIO (IT); Alessandro ROTILIO, BUSTO ARSIZIO (IT)

(21) Appl. No.: 16/483,723

(22) PCT Filed: Jan. 26, 2018
(86) PCT No.: PCT/IB2018/050488

§ 371 Date: Aug. 5, 2019

(30) Foreign Application Priority Data
Feb. 10, 2017 (IT) 102017000014692

(51) Int. Cl. A61B 6/00; A61B 6/03; G06T 7/00; G06T 3/00

(52) U.S. Cl. CPC A61B 6/5211; A61B 6/032; A61B 6/4085; G06T 2207/30061; G06T 3/0075; G06T 2207/10081; G06T 7/0014

(57) ABSTRACT

A system for processing image data from computed tomography scans has: a first detection device capable of being activated prior to an antitumor treatment on a patient and configured to perform a standard computed tomography scan on the patient to obtain first image data; a second detection device capable of being activated intraoperatively at the end of the patient's antitumor treatment and configured to perform a cone-beam computed tomography scan to obtain second image data; a processing unit, in data communication with the first device and the second device and configured to process the first image data and the second image data, thereby providing a graphic comparison between a tumor mass prior to the antitumor treatment and a necrotic area after the antitumor treatment. A corresponding method for processing image data from computed tomography scans is also described.

### FIELD OF APPLICATION

The present invention relates to a system for processing data from computed axial tomography scans.

The present invention further relates to a method for processing data from computed axial tomography scans.

In particular, the present invention relates to a system and method for processing data from computed axial tomography scans relating to antitumour treatment methods.

More in particular, the present invention relates to a system and method for processing data from computed axial tomography scans relating to methods of antitumour treatment for the liver or lungs and the present description makes reference to this field of application to simplify the illustration thereof.

### PRIOR ART

At present, data from standard computed tomography (CT) scans prior to and after surgical treatment, for example by means of ablation of the tumour mass, are compared to establish whether a supplementary antitumour treatment is necessary.

Comparisons between the two standard computed tomography (CT) scans are point comparisons aimed at evaluating the one-to-one correspondence between pre-treatment image data representing a body area in which a tumour mass is present and post-treatment image data representing the same body area from which the tumour mass was treated.

Unfortunately, in order to be reliable, the processing described requires that the second standard computed tomography scan is performed no less than two weeks after the treatment.

This results in the need, in the event that the graphic processing does not confirm a perfect outcome of the treatment, for rehospitalization and a supplementary treatment, which will be followed by a new post-treatment standard computed tomography scan for comparison.

The overall processing of pre- and post-antitumour treatment data is therefore greatly delayed over time.

This obviously results in prolonged treatment periods and consequent health risks for a patient who is not promptly treated.

Furthermore, it is not feasible to perform a standard computed tomography scan intraoperatively at the end of the patient's antitumour treatment, so as to compare it with a pre-treatment computed tomography scan, because it would require excessively complex logistics and risks for the patient's health.

In light of the above, the processing of data from computed tomography scans for antitumour treatments is presently inefficient.

The object of the present invention is to provide an efficient system and method for processing data from computed tomography scans.

A particular object of the present invention is to provide a system and method for processing data from computed tomography scans that is efficient in terms of processing overall pre- and post-antitumour treatment data.

### SUMMARY OF THE INVENTION

In a first aspect of the invention, these and other objects are achieved by a system for processing data from computed tomography scans comprising: a first detection device capable of being activated prior to an antitumour treatment on a patient and that is configured to: perform a standard computed tomography scan on a first part of a patient comprising a second part, which, in turn, comprises a tumour mass; obtain, from said standard computed tomography scan, first image data, in a first reference system, comprising:
a first portion of an image representing said first part of said patient;
a second portion of an image representing said second part of said patient;
a third portion of an image representing said tumour mass; a second detection device capable of being activated intraoperatively at the end of the patient's antitumour treatment and configured to:

perform a cone-beam computed tomography scan on a third part of said patient, wherein said third part substantially coincides with said second part and said third part comprises a necrotic area resulting from said antitumour treatment;

obtain, from said cone-beam computed tomography scan, second image data, in a second reference system, comprising:
a fourth portion an image representing said third part of said patient;
a fifth portion an image representing said necrotic area; a processing unit, in data communication with said first detection device and said second detection device, and configured to process said first image data and said second image data, and which comprises:

a first graphics processing module configured to carry out a first graphics processing step for graphically processing said first image data, thereby graphically highlighting, in said first image data, said second image portion and said third image portion;

a second graphics processing module configured to carry out a second graphics processing step to make said first image data in said first reference system graphically comparable with said second image data in said second reference system;

a third graphics processing module configured to graphically overlay said second image portion and said fourth image portion;

a calculation module configured to calculate a degree of overlap between
said third image portion, comprised in said second image portion and
said fifth image portion comprised in said fourth image portion, thereby realizing a graphic comparison between said tumour mass prior to antitumour treatment and said necrotic area after antitumour treatment.

Advantageous aspects are disclosed in dependent claims 2 to 6.

These and other objects are further achieved by a method for processing data from computed tomography scans comprising the steps:

prior to an antitumour treatment on a patient,

performing a standard computed tomography scan on a first part of a patient, said first part comprising a second part, which, in turn, comprises a tumour mass;

obtaining, from said standard computed tomography scan, first image data, in a first reference system, said first image data comprising:
a first portion of an image representing said first part of said patient;
a second portion of the image representing said second part of said patient;
a third portion of the image representing said tumour mass;

carrying out a first graphics processing step for graphically processing said first image data, thereby graphically highlighting, in said first image data, said second image portion and said third image portion;

intraoperatively, at the end of the patient's antitumour treatment,

performing a cone-beam computed tomography scan on a third part of said patient, wherein said third part substantially coincides with said second part and said third part comprises a necrotic area resulting from said antitumour treatment;

obtaining, from said cone-beam computed tomography scan, second image data in a second reference system, comprising:
a fourth portion of the image representing said third part of said patient;
a fifth portion of the image representing said necrotic area;

carrying out a second graphics processing step between said first reference system and said second reference system, thereby making said first image data and said second image data graphically comparable;

graphically overlaying said second image portion and said fourth image portion;

calculating a degree of overlap between:
said third image portion, comprised in said second image portion and
said fifth image portion comprised in said fourth image portion,

thereby realizing a graphic comparison between said tumour mass prior to antitumour treatment and said necrotic area after antitumour treatment.

Advantageous aspects are described in dependent claims 8 to 16.

In a third aspect, the method for processing data from computed tomography scans is computer implemented.

In a fourth aspect, the invention comprises a computer program which performs one or more of the steps of the processing method of the third aspect.

The invention achieves a plurality of technical effects:

efficient processing of data from computed tomography scans;

in particular, efficient processing of data from computed tomography scans in terms of overall processing of data prior to and after antitumour treatment.

consequent efficient antitumour treatment.

The technical effects/advantages cited and other technical effects/advantages of the invention will emerge in further detail from the description provided herein below of an example embodiment and it is provided by way of approximate and non-limiting example with reference to the attached drawings.

### BRIEF DESCRIPTION OF THE DRAWINGS

FIG. 1 is a schematic description of the system/method of the invention.

FIGS. 2 to 5 show graphically processed images of the system/method of the invention.

FIG. 6 is a general functional block diagram of the system of the invention.

### DETAILED DESCRIPTION

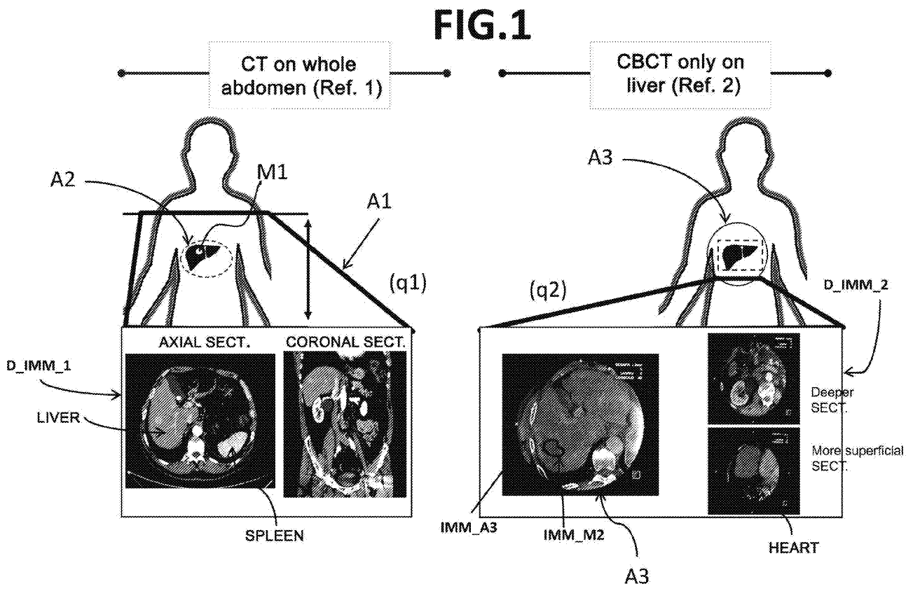

FIG. 1 is a schematic description of the system/method of the invention, which schematically shows a comparison between a technique for a standard computed tomography scan focused on the whole abdomen of a patient with respective axial and coronal cross-sectional views (on the left in the figure), and a technique for a cone-beam computed tomography scan focused on the patient's liver with respective cross-sectional views on different scanning levels (on the right in the figure).

Tomography scans are performed prior to an antitumour treatment (standard computed tomography scan) and intraoperatively at the end of the patient's antitumour treatment (cone-beam computed tomography scan).

Tomography scans are performed by two different detection devices D1, D2 and the data produced are processed by a processing unit 100.

The antitumour treatment preferably takes place by means of ablation of a tumour mass, i.e. by destruction, in particular achieved with a heated needle, of neoplastic cells circulating in the body so as to prevent the development of metastases and recurrence.

In the present invention, the applicant has considered that the detail offered by a standard computed tomography scan is greater than that offered by a cone-beam computed tomography scan. Therefore, for diagnostic purposes, i.e. in order to diagnose a tumour, a standard computed tomography scan is used; in fact, it represents, in the case of a liver tumour, a scan of the whole abdomen (or more in general of the torso) and is therefore able to study the pathology also outside the liver (lymph nodes, etc); in the case of a lung tumour, on the other hand, it represents a scan of the torso and therefore it is possible to study the pathology also outside the lung.

The applicant has further considered that, also for tumour staging purposes, i.e. in order to describe schematically how large a tumour is and how far it has extended from the original site of development, a standard computed tomography scan is necessary.

The applicant has further considered that it is neither efficient nor useful to use a cone-beam computed tomography scan for the step of diagnosing a tumour, since a cone-beam computed tomography scan is only a focused scan of one part (for example the liver or a lung) and at times has difficulty in containing the entire part.

The applicant has further verified that the contrast medium used for a cone-beam computed tomography scan is in large doses (much larger compared to a standard computed tomography scan), so it is potentially harmful to consider giving two doses of contrast medium close together, i.e. it is harmful to consider doing a cone-beam computed tomography scan prior to treatment and intraoperatively at the end of the patient's antitumour treatment.

With reference to FIG. 1, the invention discloses a processing system and method for processing image data D_IMM_1, D_IMM2 from computed tomography scans, in particular from a standard computed tomography scan CT and from a cone-beam computed tomography scan CBCT.

The invention comprises performing a standard computed tomography scan CT on a first part A1 of a patient.

In a preferred embodiment of the invention, the first part A1 of the patient is an abdominal area, in particular comprising the liver plus the surrounding areas.

Alternatively, the first part A1 can be the area of the torso, i.e. the area of the abdomen and chest taken together.

The first part A1 comprises a second part A2 of a patient.

In a preferred embodiment of the invention, the second part A2 of the patient is the area of the liver.

In an alternative embodiment, the second part A2 of the patient is the area of a lung.

The second part A2 comprises, in turn, a tumour mass M1.

With reference to FIG. 1, the invention comprises obtaining, from the standard computed tomography scan CT, the first image data D_IMM_1, in a first reference system Rif_1.

According to the invention, the standard computed tomography scan CT is performed prior to an antitumour treatment on the patient, in particular on the tumour mass M1.

A first detection device (D1), capable of being activated prior to an antitumour treatment on the patient, is configured to perform a standard computed tomography scan CT on a first part A1 of the patient, which comprises a second part A2, which, in turn, comprises the tumour mass M1.

The first detection device (D1) is further configured to obtain, from the standard computed tomography scan CT, the first image data D_IMM_1, in the first reference system Rif_1.

In particular, the first reference system Rif_1 is a Cartesian coordinate system with three dimensions (X,Y,Z), characterized by a first predetermined image quality q1.

The standard computed tomography scan CT acquires a volume of interest and the 3D data are subsequently processed and rendered on three planes: axial, coronal and sagittal.

For example, with reference to FIG. 1, the first image data D_IMM_1, which comprise the axial and coronal cross sections of the first part A1 of the patient, are shown at the bottom left.

In particular, in the figure that shows the axial cross section, one can clearly see the liver (which occupies nearly the totality of the left part of the figure) and the spleen (at the bottom right in the figure).

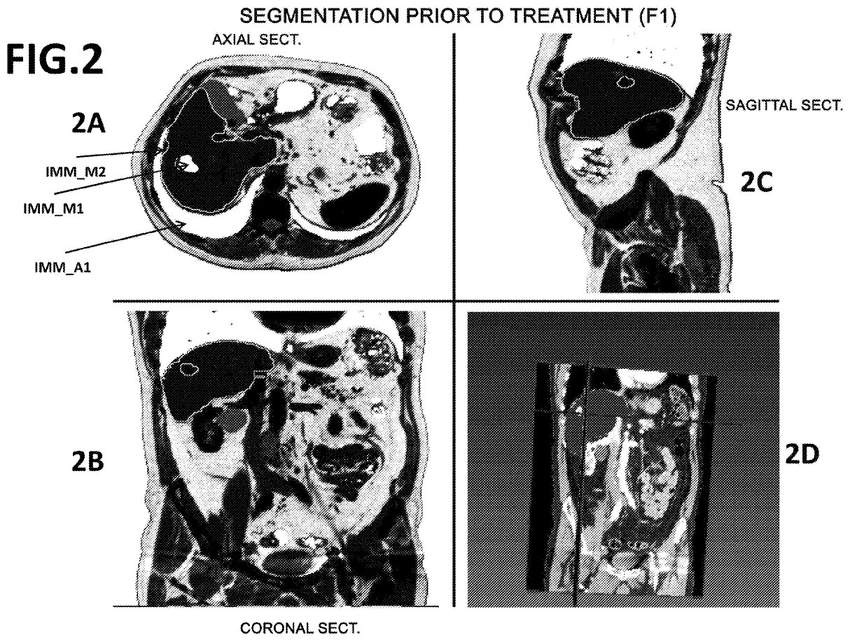

FIG. 2 shows an example of graphically processed images; in particular, the images on the left represent the outcome of a standard computed tomography scan CT in axial and coronal cross sections and coincide with those of FIG. 1 at the bottom left.

With specific reference to FIG. 2, according to the invention, the first image data D_IMM_1 comprise:
a first portion of an image IMM_A1 representing said first part A1 of said patient;
a second portion of the image IMM_A2 representing said second part A2 of said patient;
a third portion of the image IMM_M1 representing said tumour mass M1.

In particular, FIG. 2 shows a series of images obtained from the standard computed tomography scan CT prior to the antitumour treatment, according to an axial cross section 2A, a coronal cross section 2B, a sagittal cross section 2C and a cross section representing a window in which it is possible to move along three axes to inspect the volume of interest.

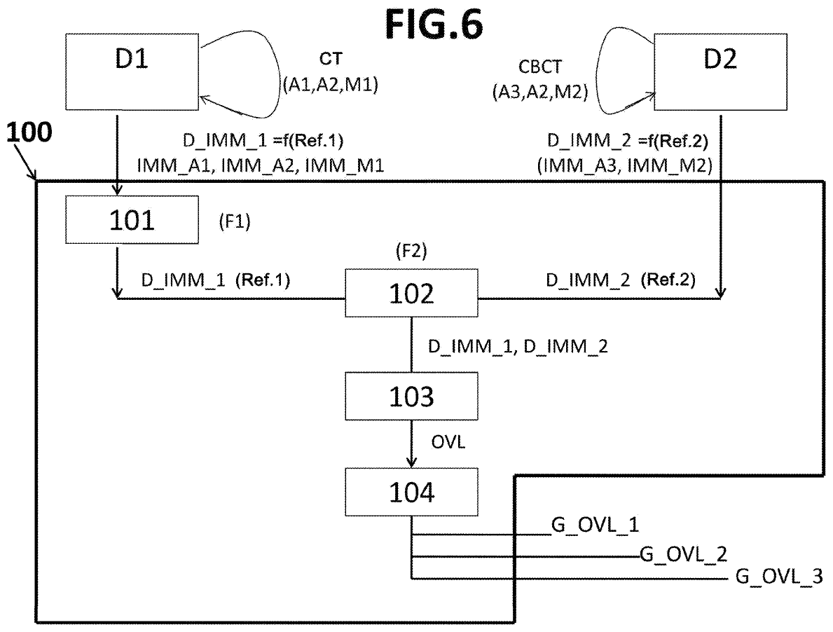

The invention comprises carrying out a first graphics processing step F1 (FIG. 6) for graphically processing the first image data D_IMM_1.

A technical effect of this processing is a graphic highlighting, in the first image data D_IMM_1, of the second image portion IMM_A2 and of the third image portion IMM_M1.

According to the invention, the first graphics processing step F1 is carried out by means of graphic segmentation of the first image data D_IMM_1.

In other words, with reference to FIG. 2, the first image data D_IMM_1 are partitioned into homogeneous regions on the basis of a certain criterion of belonging of the voxel (unit of volume) to a target region.

The technical effect is to identify/recognize the objects making up the image, so as to obtain a more compact representation for a detailed analysis of the images.

In other words, the segmentation enables the representation of the images to be simplified and/or changed into a more meaningful and/or easier-to-analyse graphic form.

The segmentation, carried out with reference to FIG. 2, enables precise identification of the area of the cross sections 2A, 2B, 2C and 2D, in which the second part A2 represented by the second image portion IMM_A2, in particular the patient's liver, is present, and in which the tumour mass M1 represented by the third image portion IMM_M1 representing the tumour mass M1 is present.

Following the described segmentation prior to the antitumour treatment, the patient undergoes an antitumour treatment intraoperatively.

The antitumour treatment preferably takes place by means of ablation of the tumour mass M1, i.e. by means of destruction, in particular achieved with a heated needle, of neoplastic cells circulating in the body so as to prevent the development of metastases and recurrence.

Intraoperatively, at the end of the patient's antitumour treatment, the invention comprises performing a cone-beam computed tomography scan CBCT on a third part A3 of the patient.

In a preferred embodiment of the invention, the third part A3 substantially coincides with the second part A2, in particular the patient's liver.

Alternatively, the third part A3, which substantially coincides with the second part A2, is a lung of the patient.

In a preferred embodiment of the invention, the third part A3 comprises a necrotic area M2 resulting from the antitumour treatment.

The invention comprises obtaining, from the cone-beam computed tomography scan CBCT, the second image data D_IMM_2 in a second reference system Rif_2 (FIG. 1).

According to the invention, the cone-beam computed tomography scan CBCT is performed intraoperatively at the end of the patient's antitumour treatment.

A second detection device D2 capable of being activated intraoperatively at the end of the patient's antitumour treatment is configured to perform the cone-beam computed tomography scan CBCT on the third part A3 of the patient, which substantially coincides with the second part A2, and comprises the necrotic area M2 resulting from said antitumour treatment.

A second detection device D2 is further configured to obtain, from the cone-beam computed tomography scan CBCT, the second image data D_IMM_2 in a second reference system Rif_2.

In particular, as schematically shown in FIG. 1 on the right, the second reference system Rif_2 is a Cartesian coordinate system with three dimensional cone-beam coordinates X,Y,Z characterized by a second predetermined image quality q2 of a lesser quality than the first predetermined image quality q1.

With specific reference to FIG. 1, according to the invention, the second image data D_IMM_2 comprise:

a fourth portion of the image IMM_A3 representing the third part A3 of the patient;

a fifth portion of the image IMM_M2 representing the necrotic area M2.

In particular, FIG. 1 shows a series of images obtained from the cone-beam computed tomography scan CBCT at the end of the patient's antitumour treatment, according to axial cross sections at a different scanning level.

As already mentioned, the invention comprises a processing unit 100, in particular with reference to FIG. 6, in data communication with the first detection device (D1) and the second detection device D2, and configured to process the first image data D_IMM_1 and the second image data D_IMM_2.

In general, it should be noted that in the present context and in the subsequent claims, the processing unit 100 is presented as being split into distinct functional modules (storage modules and operative modules) for the sole purpose of describing its functionalities clearly and completely.

The processing unit can consist of a single electronic device, appropriately programmed to perform the functionalities described, and the different modules can correspond to hardware entities and/or routine software that are part of the programmed device.

Alternatively or additionally, these functions can be performed by a plurality of electronic devices in which the above-mentioned functional modules can be distributed.

The processing unit 100 can also make use of one or more processors for execution of the instructions contained in the memory modules.

The above-mentioned functional modules can also be distributed in different computers, locally or remotely, based on the architecture of the network in which they reside.

According to the invention, the processing unit 100 comprises a first graphics processing module 101 configured to perform the first step F1 (FIG. 2) for graphically processing the first image data D_IMM_1, thereby graphically highlighting, in the first image data D_IMM_1, the second image portion IMM_A2 and the third image portion IMM_M1.

In other words, the first graphics processing module 101 carries out the graphic segmentation of the first image data D_IMM_1, previously described.

The invention comprises performing a second graphics processing step F2 (FIG. 6) between the first reference system Rif_1 and the second reference system Rif_2.

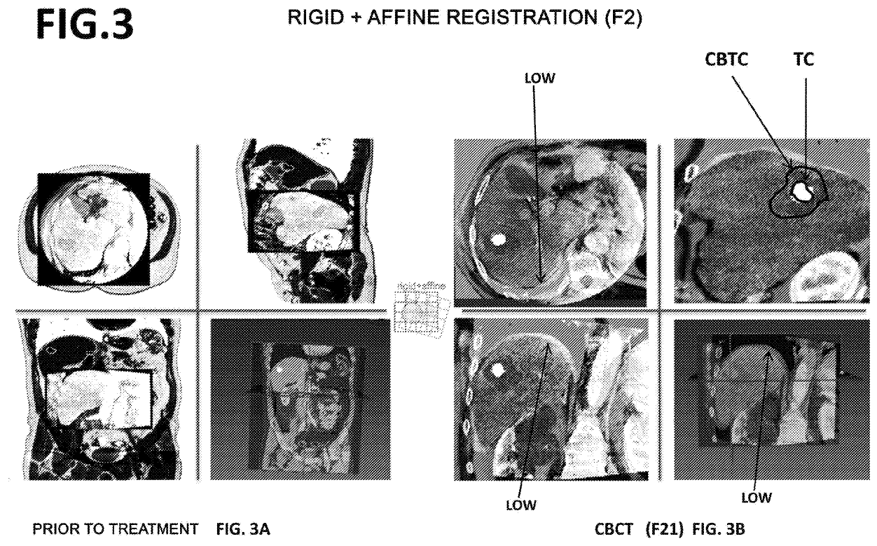

FIG. 3A shows a comparison between the initial state of the standard computed tomography scan CT, represented by the cross sections almost in their entirety, and the initial state of the cone-beam computed tomography scan CBCT, represented by the area inside the white line; as can be seen, prior to the graphics processing step F2, the two tomography scans are not comparable.

The technical effect achieved by the second graphics processing step F2 is thus to make the first image data D_IMM_1 and second image data D_IMM_2 graphically comparable.

For this purpose, the processing unit 100 comprises a second graphics processing module (102) configured to carry out a second graphics processing step F2 to make the first image data D_IMM_1 in the first reference system Rif_1 graphically comparable with the second image data D_IMM_2 in the second reference system Rif_2.

The second graphics processing step F2 is carried out by means of graphic registration of the first image data D_IMM_1, obtained with the standard computed tomography scan CT, with the second image data D_IMM_2 obtained with the cone-beam computed tomography scan CBCT.

According to the invention, with reference to FIG. 3 (images on the right), the graphic registration comprises a step F21 of rigid registration and affine registration.

The second graphics processing module 102 is configured to carry out the step of rigid registration and affine registration F21.

The technical effect is to ensure a generic graphic realignment of the first image data D_IMM_1 with the second image data D_IMM_2, wherein the realignment comprises rotation, translation and relative scaling (contraction or expansion) of the first and second image data.

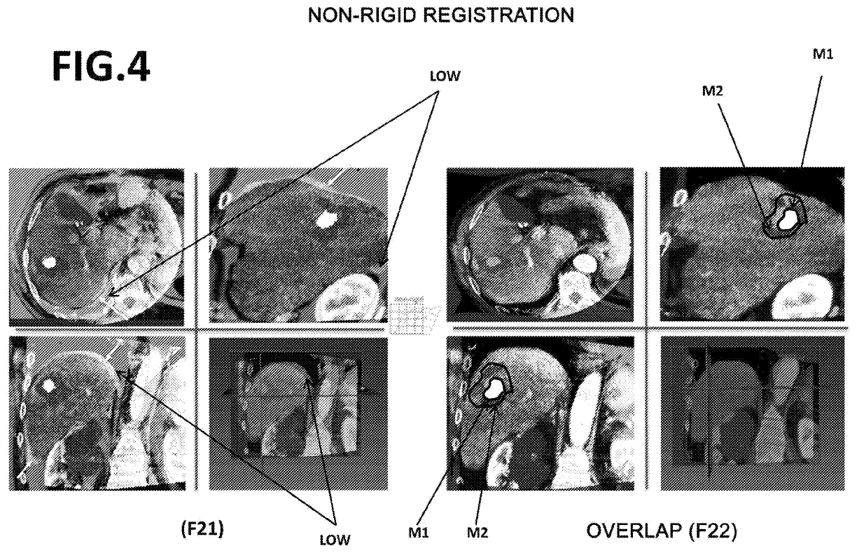

The effect is shown in FIG. 3 (or FIG. 4 in the group of images on the left), where, however, one may note low definition shadowy areas LOW where the rigid and affine registration was not able to perfectly register the first and second image data, which are not perfectly aligned.

Consequently, the “visibility” of such areas is limited, which can represent a large difficulty in identifying a possible tumour mass.

According to the invention, with reference to FIG. 4, the graphic registration further comprises a step of non-rigid registration F22.

The second graphics processing module 102 is configured to perform the non-rigid registration step F22.

The technical effect is to ensure that the first image data D_IMM_1 and the second image data D_IMM_2 are realigned so as to correct local deformations.

In other words, the non-rigid registration step F22 enables the shadowy areas LOW to be eliminated by aligning the first image data D_IMM_1 and second image data D_IMM_2, thereby also correcting deformations that are only local and do not regard the whole image; this enables maximum “visibility” of the area affected by a possible tumour.

The technical effect can be seen in FIG. 4 (group of images on the right), where it is evident that the shadowy areas LOW are no longer present.

At the end of the graphics processing step F2, which achieves a graphic registration of the first image data D_IMM_1 with the second image data D_IMM_2, the image data are comparable for subsequent processing.

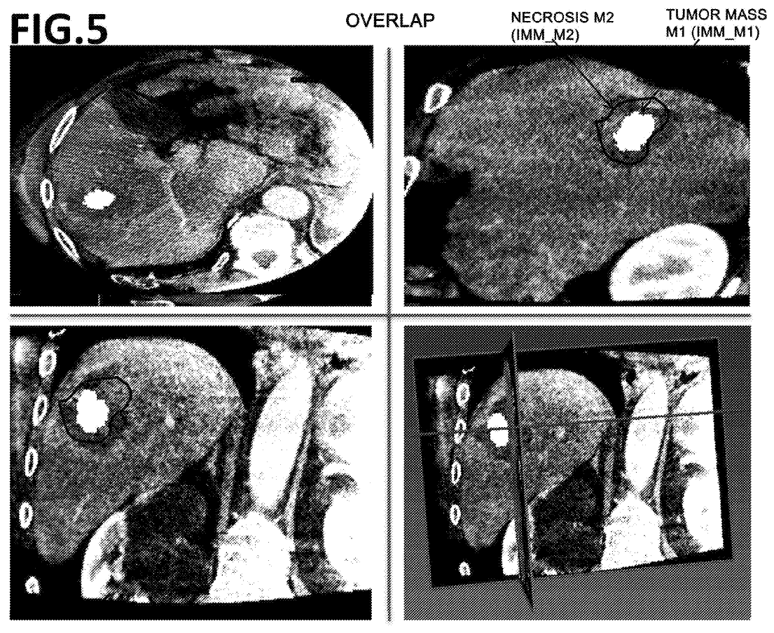

With reference to FIG. 5, the invention comprises graphically overlaying the second image portion IMM_A2 on the fourth image portion IMM_A3.

According to the invention, a third graphics processing module 103 of the processing unit 100 is configured to graphically overlay OVL the second image portion IMM_A2 and the fourth image portion IMM_A3.

In other words, in a preferred embodiment, the overlaying step comprises overlaying the image of the liver IMM_A2 produced with the standard computed tomography scan CT with the image of the liver IMM_A3 produced with the cone-beam computed tomography scan CBCT.

In an alternative embodiment, the overlaying step comprises overlaying the image of a lung IMM_A2 produced with the standard computed tomography scan CT with the image of a lung IMM_A3 produced with the cone-beam computed tomography scan CBCT.

Advantageously, the invention comprises calculating the degree of overlap G_OVL between the third image portion IMM_M1, comprised in the second image portion IMM_A2 and the fifth image portion IMM_M2 comprised in the fourth image portion IMM_A3.

A calculation module 104 of the processing unit 100 is configured to calculate the degree of overlap G_OVL.

In other words, the invention comprises making a graphic comparison between the tumour mass M1 prior to the antitumour treatment and the necrotic area M2 after the antitumour treatment.

The technical effect achieved is an evaluation of a correspondence between the limits of the tumour prior to treatment and the limits of the area that was treated, preferably by ablation, in the intraoperative phase.

According to the invention, the step of calculating the degree of overlap G_OVL determines one of the following conditions:

complete overlap G_OVL1 representing an antitumour treatment carried out successfully

partial overlap G_OVL2 representing the need to consider the advisability of further intraoperative antitumour treatment;

absence of overlap G_OVL3 representing the need for further intraoperative antitumour treatment.

The calculation module 104 (FIG. 6) is configured to calculate the degree of overlap G_OVL and determine one of the above-mentioned conditions.

The graphic registration of the invention enables the tumour prior to treatment to be compared with ablation after treatment (or during treatment, given that it is a CBCT scan), in particular in the liver or in a lung; the comparison is made on the basis of graphic processing.

The retreatment can thus be performed directly at the same operating site if it is seen that, after the treatment, the tumour area was not completely centered or was only partially centered.

Up to now, the comparison was made by performing a standard computed tomography scan the day after the treatment, and it was necessary to rehospitalize the patient and do another session in the operating room, in the event that the treatment was unsuccessful.

The method/system of the invention is thus highly efficient and protects the patient's health.

In particular, the invention also relates to a method for an antitumour treatment on the human body comprising the steps of: prior to an antitumour treatment on a patient,

performing a standard computed tomography scan CT on a first part A1 of a patient comprising a second part A2, which, in turn, comprises a tumour mass M1;

performing an antitumour treatment, preferably by means of ablation of the tumour mass M1;

intraoperatively, at the end of the patient's antitumour treatment,

performing a cone-beam computed tomography scan CBCT on a third part A3 of said patient, wherein said third part A3 substantially coincides with said second part A2 and said third part A3 comprises a necrotic area M2 resulting from said antitumour treatment;

graphically overlaying said second image portion IMM_A2 and said fourth image portion IMM_A3;

calculating a degree of overlap G_OVL between:

said third image portion IMM_M1, comprised in said second image portion IMM_A2 and

said fifth image portion IMM_M2 comprised in said fourth image portion IMM_A3

evaluating the success of the treatment according to the degree of overlap detected.

The antitumour treatment method comprises the step of ending the treatment in the case of complete overlap G_OVL1.

Alternatively, the method comprises the step of considering the advisability of further intraoperative antitumour treatment in the event of partial overlap G_OVL2.

In a further alternative, the method comprises the step of performing a further intraoperative antitumour treatment in the event of absence of overlap G_OVL3.

1.-17. (canceled)

18. A processing system for processing image data from computed tomography scans, comprising:
A) a first detection device capable of being activated prior to an antitumor treatment on a patient and that is configured to:
A1) perform a standard computed tomography scan on a first part of the patient, said first part comprising a second part, which, in turn, comprises a tumor mass;
A2) obtain, from said standard computed tomography scan, first image data in a first reference system, comprising:
a first image portion, representing said first part of said patient;
a second image portion, representing said second part of said patient; and
a third image portion, representing said tumor mass;
B) a second detection device capable of being activated intraoperatively at the end of the patient's antitumor treatment and that is configured to:
B1) perform a cone-beam computed tomography scan on a third part of said patient, wherein said third part substantially coincides with said second part and comprises a necrotic area resulting from said antitumor treatment;
B2) obtain, from said cone-beam computed tomography scan, second image data in a second reference system, said second image data comprising:
a fourth image portion, representing said third part of said patient; and
a fifth image portion, representing said necrotic area;
C) a processing unit, in data communication with said first detection device and said second detection device, configured to process said first image data and said second image data, the process unit comprising:
C1) a first graphics processing module configured to perform a first graphics processing step for graphically processing said first image data, thereby graphically highlighting, in said first image data, said second image portion and said third image portion;
C2) a second graphics processing module configured to perform a second graphics processing step to make said first image data in said first reference system graphically comparable with said second image data in said second reference system;
C3) a third graphics processing module configured to graphically overlay said second image portion and said fourth image portion; and
C4) a calculation module configured to calculate a degree of overlap between:
said third image portion, comprised in said second image portion and
said fifth image portion comprised in said fourth image portion, thereby providing a graphic comparison between said tumor mass prior to the antitumor treatment and said necrotic area after the antitumor treatment.

19. The processing system according to claim 18, wherein said first graphics processing module is configured to perform said first graphics processing step by graphic segmentation of said first image data.

20. The processing system according to claim 18, wherein said calculation module is configured to calculate the degree of overlap and to determine one of:
complete overlap, representing a successfully performed antitumor treatment;
partial overlap, representing a need to consider advisability of further intraoperative antitumor treatment; and
absence of overlap, representing a need for further intraoperative antitumor treatment.

21. The processing system according to claim 20, wherein said second graphics processing module is configured to perform said second graphics processing step by graphic registration of:
said first image data, obtained with said standard computed tomography scan with
said second image data, obtained with said cone-beam computed tomography scan.

22. The processing system according to claim 21, wherein said second graphics processing module is configured to perform, in said graphic registration:
a step of rigid registration and affine registration, thereby performing a generic graphic realignment of said first image data with said second image data, said realignment comprising rotation, translation and relative scaling of the first and second image data; and
a step of non-rigid registration, thereby performing a precise graphic realignment of said first image data with said second image data, to correct local deformations.

23. The processing system according to claim 18, wherein said first part of the patient is a torso area and said second part is a liver or lung area.

24. A computer-implemented method for processing image data from computed tomography scans, comprising the steps of:
prior to an antitumor treatment on a patient,
A) performing a standard computed tomography scan on a first part of the patient, said first part comprising a second part, which, in turn, comprises a tumor mass;
B) obtaining, from said standard computed tomography scan, first image data in a first reference system, said first image data comprising:
a first image portion, representing said first part of said patient;
a second image portion, representing said second part of said patient; and
a third image portion, representing said tumor mass;
C) performing a first graphics processing step for graphically processing said first image data, thereby graphically highlighting, in said first image data, said second image portion and said third image portion;
intraoperatively, at the end of the antitumor treatment on the patient,
D) performing a cone-beam computed tomography scan on a third part of said patient, wherein said third part substantially coincides with said second part and said third part comprises a necrotic area resulting from said antitumor treatment;
E) obtaining, from said cone-beam computed tomography scan, second image data in a second reference system, said second image data comprising:
a fourth image portion, representing said third part of said patient; and
a fifth image portion, representing said necrotic area;
F) performing a second graphics processing step between said first reference system and said second reference system, thereby making said first image data and said second image data graphically comparable;
G) graphically overlaying said second image portion and said fourth image portion;
H) calculating a degree of overlap between:
said third image portion comprised in said second image portion and
said fifth image portion comprised in said fourth image portion, thereby providing a graphic comparison between said tumor mass prior to the antitumor treatment and said necrotic area after the antitumor treatment.

25. The computer-implemented method according to claim 24, wherein:
said first reference system is a Cartesian coordinate system with three dimensions, having a first image quality; and
said second reference system is a Cartesian coordinate system with three-dimensional cone-beam coordinates, having a second image quality lower than the first image quality.

26. The computer-implemented method according to claim 24, wherein said step of calculating the degree of overlap determines one of:
complete overlap, representing a successfully performed antitumor treatment;
partial overlap, representing a need to consider advisability of further intraoperative antitumor treatment; and
absence of overlap, representing a need for further intraoperative antitumor treatment.

27. The computer-implemented method according to claim 24, wherein said first graphics processing step is performed by graphic segmentation of said first image data.

28. The computer-implemented method according to claim 24, wherein said second graphics processing step is performed by graphic registration of:
said first image data, obtained with said standard computed tomography scan with
said second image data, obtained with said cone-beam computed tomography scan.

29. The computer-implemented method according to claim 28, wherein said graphic registration comprises:
a step of rigid registration and affine registration, thereby performing a generic graphic realignment of said first image data with said second image data, said realignment comprising rotation, translation and relative scaling of the first and second image data; and
a step of non-rigid registration, thereby performing a precise graphic realignment of said first image data with said second image data, so as to correct local deformations.

30. The computer-implemented method according to claim 24, wherein said antitumor treatment takes place by ablation of said tumor mass.

31. The computer-implemented method according to claim 24, wherein said first part of the patient is a torso area and wherein said second part is a liver or lung area.

32. A non-transitory computer-readable medium configured to perform, when running on a computer, the steps of the computer-implemented method according to claim 24.
